(19) United States
(12) Patent Application Publication
(10) Pub. No.: US 2026/0114861 A1
(43) Pub. Date: Apr. 30, 2026

(54) ANCHOR INSERTER SYSTEM

(71) Applicant: Arthrex, Inc., Naples, FL (US)

(72) Inventors: Peter J. Dreyfuss, Naples, FL (US); Anup Shah, Phoenix, AZ (US)

(73) Assignee: Arthrex, Inc., Naples, FL (US)

(21) Appl. No.: 19/360,655

(22) Filed: Oct. 16, 2025

Related U.S. Application Data
(60) Provisional application No. 63/711,408, filed on Oct. 24, 2024.

(51) Int. Cl. A61B 17/04

(52) U.S. Cl. CPC A61B 17/0401; A61B 2017/0409; A61B 2017/0414; A61B 2017/042; A61B 2017/0445

(57) ABSTRACT

An anchor inserter system includes an inserter having a proximal end portion and a distal end portion. The distal end portion includes a first tine and a second tine opposite the first tine. The second tine is cannulated. A slot is defined between the first tine and the second tine.

### CROSS-REFERENCE TO RELATED APPLICATION

This application claims the benefit of and priority to U.S. Provisional Application No. 63/711,408 filed on Oct. 24, 2024. The disclosure of the above application is incorporated herein by reference.

### BACKGROUND

The present disclosure relates to medical devices for the insertion of anchors that facilitate the reattachment of soft tissue to bone, and more particularly relates to an anchor inserter system.

Soft tissue, such as a ligament or tendon, may become detached from a bone. Surgery may be required to reattach the soft tissue to the bone to promote healing of the soft tissue and corresponding joint(s). In certain instances, the surgery may be performed arthroscopically, using small incisions to access a region within an anatomy of a patient proximate the corresponding joint(s).

### SUMMARY

This section provides a general summary of the disclosure and is not a comprehensive disclosure of its full scope or all of its features.

The present disclosure provides an anchor inserter system, which may permit the insertion of an anchor to a predetermined location within the patient's anatomy. The anchor inserter system may generally include a distal end, which may be coupled to the anchor for inserting the anchor into the anatomy. In one example, the distal end may be forked or may define a U-shaped slot. A portion of the anchor may be received within the U-shaped slot. A guidewire may be used with the anchor inserter system to guide the anchor to a predetermined position within the anatomy.

In one example, the anchor inserter system may include a cannulated tine, which may slidably receive the guidewire to direct a soft anchor to the predetermined location within the patient's body. The cannulated tine may cooperate with a solid or non-cannulated tine to define the fork or U-shaped slot at the distal end of the anchor inserter system. Generally, the cannulated tine may be opposite the non-cannulated tine on the sides of the U-shaped slot. A portion of the soft anchor may be received within the U-shaped slot so as to be positioned between the cannulated tine and the non-cannulated tine. The cannulated tine may slidably receive the guidewire to direct the soft anchor to the predetermined location within the anatomy.

In a further example, the anchor inserter system may be a self-punching inserter. Generally, the self-punching anchor inserter system may permit the soft anchor to be directly inserted into the bone without creating a pre-drilled or pre-punched bone hole. In this example, the cannulated tine may slidably receive the guidewire to direct the anchor inserter system to the predetermined location, and the cannulated and non-cannulated tine may cooperate to define a bone hole in the anatomy for receipt of the soft anchor. In other examples, the anchor inserter system may be used with a pre-drilled or pre-punched bone hole, in which the guidewire is positioned within the bone hole prior to the introduction of an inserter of the anchor inserter system.

In addition, it should be noted that while the above description refers to the use of the anchor inserter system with a soft anchor, the anchor inserter system may also be used with hard anchors. In one example, the anchor inserter system may be employed for a rotator cuff repair, shoulder arthroplasty, etc.

In one example, an anchor inserter system may include an inserter having a proximal end portion and a distal end portion. The distal end portion may include a first tine and a second tine opposite the first tine. The second tine may be cannulated. A slot may be defined between the first tine and the second tine.

The anchor inserter system may include an anchor, and a portion of the anchor may be coupled within the slot. The anchor may be a soft anchor. The slot may be U-shaped. The first tine may be solid. The second tine may define a tine bore extending along an axis parallel to and offset from a longitudinal axis of the inserter. The anchor inserter system may include a guidewire, and the guidewire may be slidably received within the second tine. The anchor inserter system may include a collar coupled about a portion of the guidewire. The inserter may include a graspable portion coupled to the proximal end portion and a shaft interconnecting the proximal end portion and the distal end portion, and the collar may be movable between a first position in which the collar may be proximate the graspable portion to couple the guidewire to the shaft and a second position in which the collar may be spaced apart from the graspable portion. The first tine may extend for a first length axially along a longitudinal axis of the inserter, the second tine may extend for a second length axially along the longitudinal axis of the inserter, and the second length may be greater than the first length.

Also provided is an anchor inserter system that may include a soft anchor and an inserter. The inserter may have a proximal end portion and a distal end portion. The distal end portion may include a first tine and a second tine opposite the first tine. The second tine may be cannulated. A slot may be defined between the first tine and the second tine, and a portion of the soft anchor may be received within the slot.

The slot may be U-shaped. The first tine may be solid. The second tine may define a tine bore extending along an axis parallel to and offset from a longitudinal axis of the inserter. The anchor inserter system may include a guidewire, and the guidewire may be slidably received within the second tine. The anchor inserter system may include a collar having an interior surface opposite an exterior surface, and the interior surface may be coupled about a portion of the guidewire. A second portion of the soft anchor may extend from the portion along the exterior surface of the collar to the proximal end portion. The inserter may include a graspable portion coupled to the proximal end portion and a shaft interconnecting the proximal end portion and the distal end portion, and the collar may be movable between a first position in which the collar is proximate the graspable portion to couple the guidewire to the shaft and a second position in which the collar may be spaced apart from the graspable portion.

Further provided is an anchor inserter system that may include a soft anchor having a sheath and a flexible strand, and an inserter. The inserter may have a proximal end portion and a distal end portion interconnected by a shaft. The proximal end portion may include a graspable portion. The distal end portion may include a first tine and a second tine opposite the first tine. The second tine may be cannulated, and a slot may be defined between the first tine and the second tine. The sheath may be coupled within the slot. The anchor inserter system may include a collar. The collar may have an interior surface coupled about a portion of the shaft and an exterior surface. A portion of the flexible strand may extend along the exterior surface of the collar such that the collar may be movable relative to the soft anchor.

The anchor inserter system may include a guidewire, the collar may be coupled about a portion of the guidewire and the guidewire may be slidably received within the second tine.

Further areas of applicability will become apparent from the description provided herein. It should be understood that the description and specific examples are intended for purposes of illustration and are not intended to limit the scope of the present disclosure.

### BRIEF DESCRIPTION OF THE DRAWINGS

In order that the disclosure may be well understood, there will now be described various forms thereof, given by way of example, reference being made to the accompanying drawings, in which:

FIG. 1 is a side view of a kit including an anchor inserter system in which a collar is in a first collar position.

FIG. 2 is a perspective view of a distal end of the anchor inserter system with the guidewire removed.

FIG. 3 is a perspective view of the distal end of an inserter of the anchor inserter system.

FIG. 4 is a top view of the distal end of the inserter.

FIG. 5 is detail perspective view of the distal end of the inserter taken at 5 on FIG. 3.

FIG. 6 is a perspective view of the distal end of the anchor inserter system with the guidewire advanced.

FIG. 7 is a side view of the anchor inserter system in which the collar is in a second collar position.

FIGS. 8A and 8B are simplified schematic environmental illustrations of an exemplary method for deploying the anchor using the anchor inserter system.

FIGS. 9A-9F are simplified schematic environmental illustrations of another exemplary method for deploying the anchor using the anchor inserter system.

The drawings described herein are for illustration purposes and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features. In addition, there is no intention to be bound by any expressed or implied theory presented in the preceding introduction, brief summary or the following detailed description. Further, the present disclosure may be practiced in conjunction with any type of implant that would benefit from guidance with a guidewire, and the use of the anchor inserter system to guide an anchor, such as a suture anchor, with a guidewire described herein is merely one example according to the present disclosure. Further, it should be noted that many alternative or additional functional relationships or physical connections may be present in an example of the present disclosure. In addition, while the figures shown herein depict an example with certain arrangements of elements, additional intervening elements, devices, features, or components may be present. It should also be understood that the drawings are merely illustrative and may not be drawn to scale.

As used herein, the term “axial” refers to a direction that is generally parallel to or coincident with an axis of rotation, axis of symmetry, or centerline of a component or components. For example, in a cylinder or disc with a centerline and generally circular ends or opposing faces, the “axial” direction may refer to the direction that generally extends in parallel to the centerline between the opposite ends or faces. In certain instances, the term “axial” may be utilized with respect to components that are not cylindrical (or otherwise radially symmetric). For example, the “axial” direction for a rectangular housing containing a rotating shaft may be viewed as a direction that is generally parallel to or coincident with the rotational axis of the shaft. Furthermore, the term “radially” as used herein may refer to a direction or a relationship of components with respect to a line extending outward from a shared centerline, axis, or similar reference, for example in a plane of a cylinder or disc that is perpendicular to the centerline or axis. In certain instances, components may be viewed as “radially” aligned even though one or both of the components may not be cylindrical (or otherwise radially symmetric). Furthermore, the terms “axial” and “radial” (and any derivatives) may encompass directional relationships that are other than precisely aligned with (e.g., oblique to) the true axial and radial dimensions, provided the relationship is predominantly in the respective nominal axial or radial direction. As used herein, the term “about” denotes within 15% to account for manufacturing tolerances. In addition, the term “substantially” denotes within 15% to account for manufacturing tolerances.

The present disclosure provides an anchor inserter system, which may be used in an orthopedic surgical procedure involving the fixation of soft tissue to bone. The anchor inserter system may generally include a distal end, which may be coupled to an anchor for inserting the anchor into the anatomy. In one example, the distal end may be forked or may define a U-shaped slot. A portion of the anchor may be received within the U-shaped slot. In this example, the anchor inserter system may permit the introduction of the anchor to a predetermined location within the patient's anatomy using a guidewire, which improves accuracy and enables the surgeon to insert the anchor without a clear line of sight.

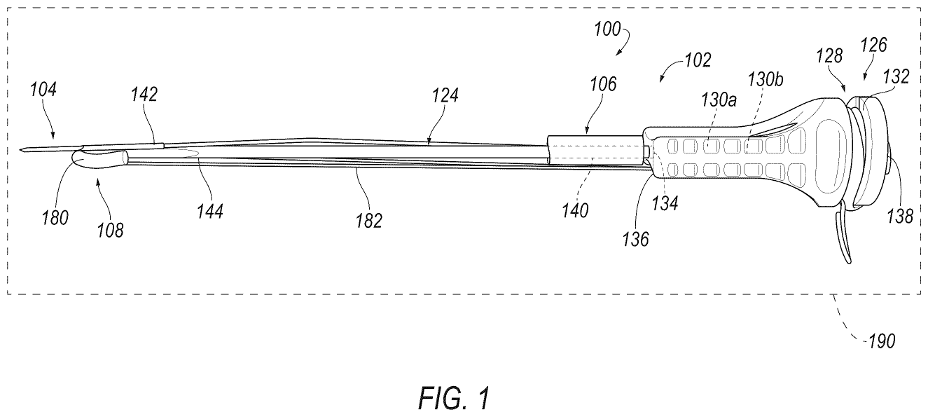

With reference to FIG. 1, an anchor inserter system 100 is shown. In one example, the anchor inserter system 100 may include an inserter 102, a guidewire 104, a collar 106 and an anchor 108. As will be discussed, the anchor inserter system 100 enables the insertion of the anchor 108 into the anatomy, and the anchor 108 and the inserter 102 may be guided into the predetermined location along the guidewire 104. In other examples, the guidewire 104 may be positioned initially, and the inserter 102 may be directed over the guidewire 104. In either example, the use of the guidewire 104 with the inserter 102 may permit the surgeon to position the guidewire 104 in the predetermined location, before directing the inserter 102 with the anchor 108 to that location for the insertion of the anchor 108. This may reduce impact to surrounding tissue during placement of the anchor 108. Moreover, the use of the guidewire 104 with the inserter 102 may allow the surgeon to position the anchor 108 in the anatomy without a clear line of sight.

The inserter 102 may deliver the anchor 108 to the predetermined location within the anatomy. In one example, the inserter 102 may include a proximal end portion or proximal end 120 opposite a distal end portion or distal end 122, and a shaft 124 that may interconnect the proximal end 120 with the distal end 122. The proximal end 120 may include a manipulatable portion, graspable portion or handle 126 and a suture retainer 128. The handle 126 may be composed of a polymer-based material, however, any suitable biocompatible material may be employed. In one example, the handle 126 may include at least one graspable slot 130, a suture retainer groove 132 and a handle bore 134. In this example, the at least one graspable slot 130 includes a pair of graspable slots 130a, 130b that may be defined through the handle 126. The graspable slots 130a, 130b may be elongated and substantially oval in shape to enable one or more fingers of the surgeon to manipulate the inserter 102. In one example, the graspable slots 130a, 130b may be spaced apart from each other by a portion of the handle 126 that defines the handle bore 134. Generally, the graspable slots 130a, 130b may be defined through the handle 126 so as to be proximate a distal handle end 136, with the distal handle end 136 opposite a proximal handle end 138.

The suture retainer groove 132 may be defined about a perimeter of the handle 126. Generally, the suture retainer groove 132 may be defined through the handle 126 so as to be in communication with the handle bore 134. The suture retainer groove 132 may be sized to receive and retain the suture retainer 128 such that the suture retainer 128 may be coupled to suture retainer groove 132. The suture retainer groove 132 may be defined through the handle 126 so as to be positioned between the graspable slots 130a, 130b and the proximal handle end 138 to provide ease of access to the suture retainer 128.

The handle bore 134 may be defined through the handle 126 from the distal handle end 136 toward the proximal handle end 138. The handle bore 134 may receive the shaft 124 to couple the handle 126 to the shaft 124. The shaft 124 may be coupled to the handle 126 in any suitable manner, including, but not limited to, press-fit, overmolding, mechanical fasteners, etc. Generally, the portion of the anchor 108 may extend along a surface of the handle 126 and be secured within the suture retainer groove 132 via the suture retainer 128. Alternatively, the handle bore 134 may be sized to enable a portion of the anchor 108 to extend through the handle bore 134, along the shaft 124, and be coupled to the suture retainer 128 via the suture retainer groove 132.

The suture retainer 128 may be removably coupled to the suture retainer groove 132. Generally, when coupled to the suture retainer groove 132 and the handle 126, the suture retainer 128 may retain the anchor 108 on the inserter 102. When the suture retainer 128 is removed from or uncoupled from the handle 126, the anchor 108 may be released from inserter 102. In one example, the suture retainer 128 may be substantially circular and may include a pull tab. The pull tab may provide a graspable portion for the surgeon to enable the surgeon to easily remove the suture retainer 128 to release the anchor 108 from the suture retainer 128. The suture retainer 128 may be composed of any suitable biocompatible material, and for example, may be composed of a biocompatible polymer-based material.

The shaft 124 may extend between the proximal end 120 and the distal end 122. The shaft 124 may be composed of a biocompatible material, such as a biocompatible metal or metal alloy. In one example, the shaft 124 may include a first laser mark 140 proximate the distal handle end 136 of the handle 126. The shaft 124 may include a second laser mark 142 proximate the distal end 122, with a third laser mark 144 spaced apart proximally from the second laser mark 142. The first laser mark 140, the second laser mark 142 and the third laser mark 144 may provide visual indicators as to the position of the inserter 102 and thus, the anchor 108, during a surgical procedure. In this example, the first laser mark 140 may visually indicate that the inserter 102 is in an insertion zone for the deployment of the anchor 108 when the inserter 102 is inserted through a cannula or sheath, for example. The second laser mark 142 may visually indicate that the inserter 102 may be in an insertion zone for the deployment of the anchor 108 when the inserter 102 is inserted without a cannula or a sheath. The third laser mark 144 may extend axially along the shaft 124 for a distance that may be different and less than a distance the second laser mark 142 may extend axially along the shaft 124 to visually distinguish between the second laser mark 142 and the third laser mark 144.

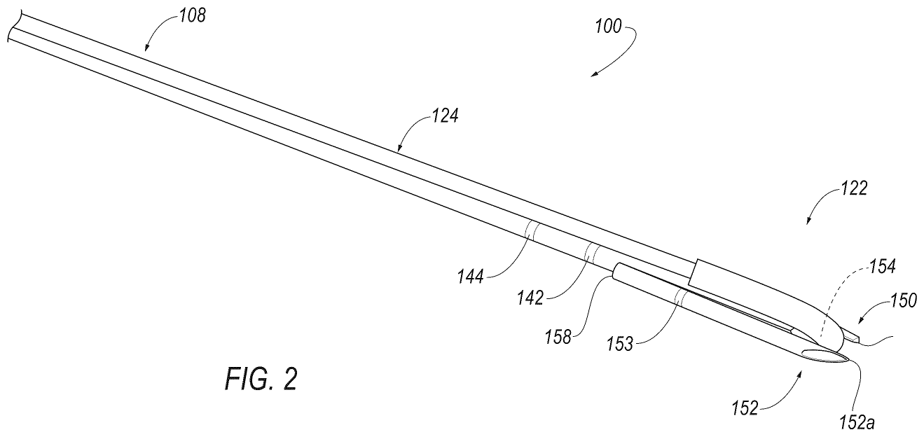

The distal end 122 may be coupled to the shaft 124 proximate the second laser mark 142. The distal end 122 may be integrally formed with the shaft 124 or all or a portion of the distal end 122 may be discretely formed and coupled to the shaft 124. In one example, with reference to FIG. 2, the distal end 122 may be forked and may include a first tine 150 opposite a second tine 152. The first tine 150 and the second tine 152 may cooperate to define a saddle or U-shaped slot 154, which may receive a portion of anchor 108. The first tine 150 and the second tine 152 may be disposed on opposite sides of the slot 154. The first tine 150 and the second tine 152 may each include a respective tip 150a, 152a. In this example, the tips 150a, 152a may be pointed or taper to a point to permit the inserter 102 to be self-punching or able to define a bone hole in the anatomy without the use of a drill. In other examples, the tip 150a, 152a of the first tine 150 and the second tine 152, respectively, may include a rounded tip for use in a pre-formed or pre-drilled bone hole.

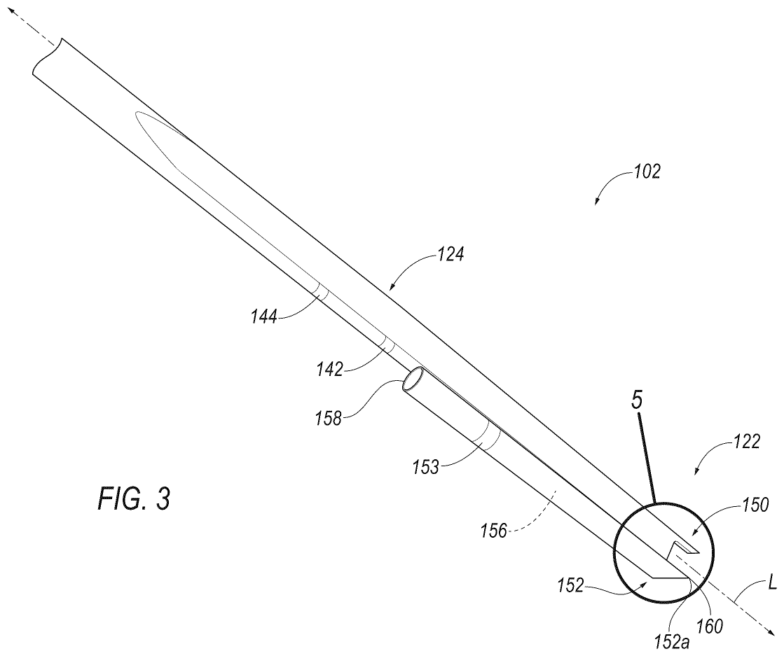

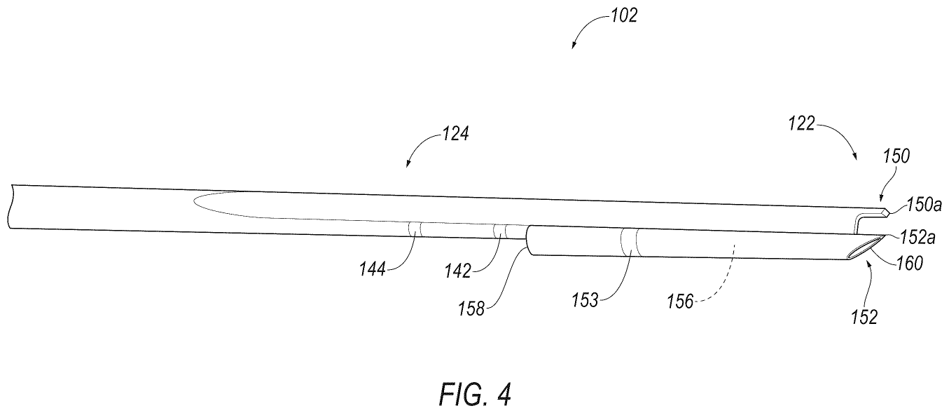

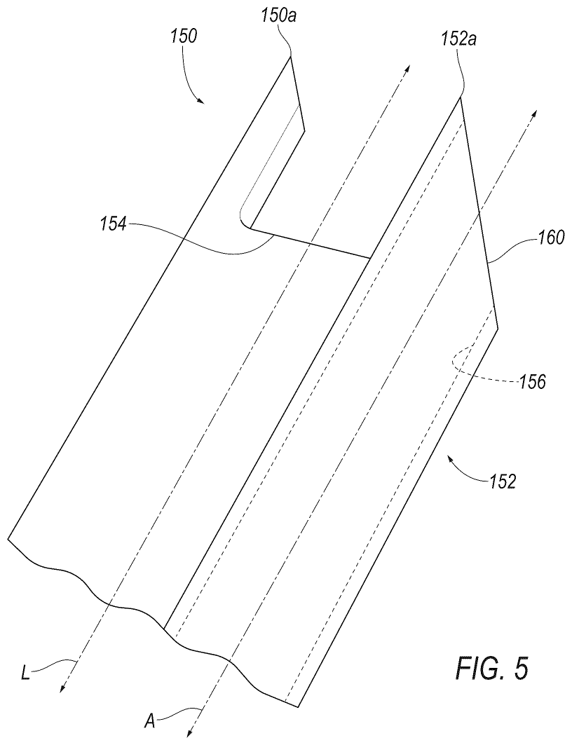

With reference to FIGS. 3-5, the distal end 122 is shown with the anchor 108 removed. In one example, the first tine 150 may be integrally formed with the shaft 124 to extend distally from the shaft 124 and the second tine 152 may be discretely formed and coupled to the shaft 124 opposite the first tine 150. For example, the second tine 152 may be coupled to the shaft 124 via welding. The second tine 152 may also include a tine laser mark 153 (FIG. 2) to visually distinguish the second tine 152 from the first tine 150. Generally, the first tine 150 may be solid or non-cannulated, and the second tine 152 may be hollow or cannulated. Stated another way, the second tine 152 may define a tine bore 156 (FIG. 5) that extends from a proximal tine end 158 to the tip 152a at a second tine end 160, with the second tine end 160 opposite proximal tine end 158. The tine bore 156 may be sized and shaped to slidably receive the guidewire 104 to enable the inserter 102 to be directed by the guidewire 104. Thus, in this example, a single tine of the first tine 150 and the second tine 152 may be cannulated.

Generally, with reference to FIG. 5, the tine bore 156 may be defined to extend along an axis A, which may be substantially parallel to and offset from a longitudinal axis L of the inserter 102. Stated another way, the tine bore 156 may extend along the axis A, which may be spaced apart from and not coaxial with the longitudinal axis L. By providing the tine bore 156 offset from the longitudinal axis L, the guidewire 104 may not interfere with and may be spaced apart from the anchor 108. Moreover, by defining the tine bore 156 offset from the longitudinal axis L, it may be less likely that the guidewire 104 may contact the anchor 108 or become entangled with a portion of the anchor 108 during insertion. Generally, when the anchor 108 is coupled to the inserter 102, the anchor 108 may extend along the longitudinal axis L.

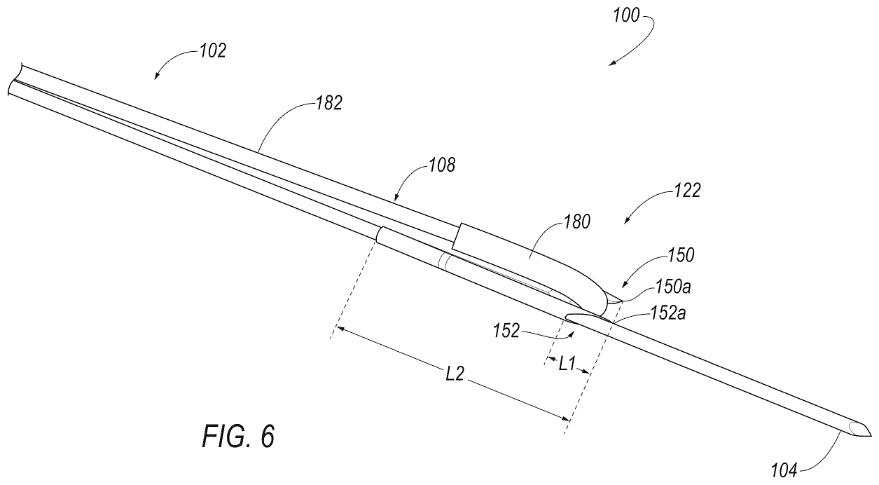

In one example, with reference to FIG. 6, the second tine 152 may extend for a second length L2 axially along the longitudinal axis L, which may be different and greater than a first length L1 the first tine 150 may extend along the longitudinal axis L. By providing second tine 152 with the greater second length L2, the second tine 152 may provide rigidity to the guidewire 104 during movement of the inserter 102 over the guidewire 104 while allowing the guidewire 104 to bend to enable placement of the anchor 108 within a center of a bone hole.

With reference back to FIGS. 3-5, the slot 154 may be defined between the first tine 150 and the second tine 152. The slot 154 may enable a portion of the anchor 108 to be received on and about the distal end 122 of the inserter 102. The slot 154 may generally have a concave curvature between the first tine 150 and the second tine 152 to provide a smooth contact surface for the portion of the anchor 108. It should be noted that although the slot 154 may be described and illustrated herein as comprising a substantially U-shape, the slot 154 may have any desired shape to receive the portion of the anchor 108.

With reference back to FIG. 1, the guidewire 104 may be sized to be slidably received within the tine bore 156. The guidewire 104 may be composed of a biocompatible material, such as a biocompatible metal or metal alloy, and may be generally flexible. In this example, the guidewire 104 may be cylindrical, and may be substantially solid or non-cannulated. The guidewire 104 may include a point at a distalmost end to assist in inserting the guidewire 104 into the anatomy.

The collar 106 may assist in positioning the guidewire 104 relative to the inserter 102. In one example, the collar 106 may be substantially cylindrical and hollow. The collar 106 may have a diameter that is sized such that the collar 106 may be positionable about the guidewire 104 and the shaft 124. Generally, the collar 106 may be coupled about the guidewire 104 and the shaft 124 so that a portion of the anchor 108 may pass along an exterior surface 170 of the collar 106. Stated another way, the collar 106 may not be positioned over the anchor 108. The collar 106 may include the exterior surface 170 and an opposite interior surface 172. The exterior surface 170 may be graspable by the surgeon to enable the surgeon to move the collar 106 along the shaft 124. The interior surface 172 may be coupled to and in contact with the guidewire 104 and the shaft 124. The collar 106 may be composed of a biocompatible material, such as a biocompatible polymer-based material.

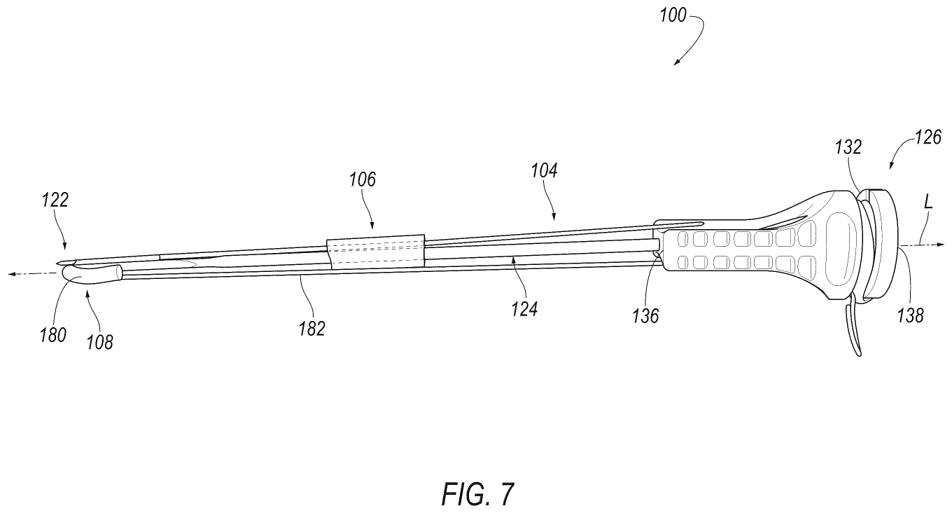

In a first collar position shown in FIG. 1, the collar 106 may be coupled to the shaft 124 and the guidewire 104 to retain the guidewire 104 on the shaft 124 so that upon removal of the inserter 102, the guidewire 104 may also be removed with the inserter 102 from the anatomy. In the first collar position, the collar 106 may be proximate or in contact with the distal handle end 136, and the guidewire 104 may be held against the handle 126. With reference to FIG. 7, in a second collar position, the collar 106 has been moved, slid or advanced axially by the surgeon toward the distal end 122 and may be spaced a distance apart from the handle 126. In the second collar position, the guidewire 104 may be deflected radially away from the handle 126 to enable the surgeon to manipulate the handle 126 to position the inserter 102 while inhibiting contact with the guidewire 104. Thus, generally, the collar 106 may be movable between a first position or the first collar position and a second position, or the second collar position.

The anchor 108 may be coupled to the inserter 102 for insertion into the anatomy. In one example, the anchor 108 may be a soft anchor. The anchor 108 may be referred to herein as “soft” because it may be formed of soft materials such as biocompatible yarns, fibers, filaments, strings, fibrils, strands, sutures, etc., or any combination of such materials. The soft materials may be synthetic or natural materials, or combinations of synthetic and natural materials, and may be bio-degradable or non-degradable within the scope of the present disclosure. For example, the anchor 108 may be a Fibertak® RC Soft Anchor commercially available from Arthrex, Inc. of Naples, Florida, USA. It should be noted, however, that any soft anchor may be employed.

In this example, the anchor 108 may include a sheath 180 and a flexible strand 182. Generally, the sheath 180 may be a tubular sleeve made of a flexible material, such as a braided, woven, or knitted structure made of biocompatible yarns, fibers, filaments, sutures or similar materials, or combinations of these materials. The sheath 180 may be free of barbs or protrusions and may be deformable to secure the anchor 108 to the anatomy. The sheath 180 may include a throughbore that receives the flexible strand 182, and may include at least one splice point or opening, which may enable the flexible strand 182 to exit and re-enter into the throughbore. Generally, the sheath 180 may be coupled to or received within the slot 154 so as to be positioned between the first tine 150 and the second tine 152.

The flexible strand 182 may be coupled to the sheath 180. In one example, the flexible strand 182 may be a suture, including, but not limited to FiberWire®, TigerWire®, or FiberChain® suture commercially available from Arthrex, Inc. of Naples, Florida, USA, although any type of suture may be utilized. The flexible strand 14 may also comprise suture tape, such as FiberTape® commercially available from Arthrex, Inc. of Naples, Florida, USA. Generally, the flexible strand 182 may comprise any soft, biocompatible flexible strand of material.

Generally, the flexible strand 182 may be coupled to the sheath 180 such that applying tension to the flexible strand 182 causes the sheath 180 to substantially bunch together into a configuration that secures the sheath 180 in the bone hole formed in the anatomy. In the bunched together configuration of the sheath 180, the anchor 108 may be deployed and may resist applied forces so that the surgeon can use the flexible strand 182 to secure soft tissue to the bone to promote healing.

With reference back to FIG. 1, the anchor inserter system 100, the guidewire 104, the collar 106 and the anchor 108 may be packaged together in a kit 190, for example. In one example of the kit 190, the guidewire 104 may be slidably received within the second tine 152 and may be coupled to the inserter 102 via the collar 106. In other examples, the guidewire 104 may be packaged separately.

An example method using the inserter 102 to fix soft tissue to bone to promote healing will be described below. It should be noted that the surgeon may or may not perform the method in the order shown and may perform the method partially or entirely. Moreover, the use of “surgeon” herein is meant to encompass other suitably trained staff that may assist with the method and use of the inserter 102. The method described below may be performed during a surgical procedure.

Initially, the shaft 124 of the inserter 102 may be assembled such that the second tine 152 may be opposite the first tine 150 to define the slot 154. With the handle 126 coupled to the inserter 102, the anchor 108 may be coupled to the inserter 102 such that the sheath 180 may be disposed within the slot 154, and the flexible strand 182 may be coupled to the suture retainer groove 132. The suture retainer 128 may be coupled about the suture retainer groove 132 to retain the flexible strand 182 and thus the anchor 108 on the inserter 102.

In one example, with reference to FIG. 7, the guidewire 104 may be slidably coupled to the second tine 152 such that a portion of the guidewire 104 passes through the tine bore 156. The collar 106 may be positioned about the inserter 102 such that the interior surface 172 of the collar 106 may contact a portion of the shaft 124 and the guidewire 104, but may not contact the flexible strand 182. Generally, the flexible strand 182 may pass along the exterior surface 170 of the collar 106 to enable the collar 106 to move without contacting the anchor 108. The collar 106 may be placed in the second collar position so that the guidewire 104 does not interfere with the handle 126.

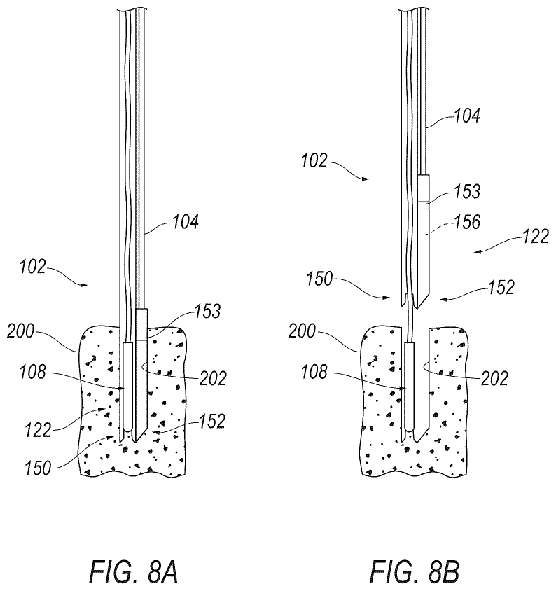

With the anchor inserter system 100 assembled, the anchor inserter system 100 may be inserted percutaneously through the anatomy, proximate the joint to be repaired. In the example of the inserter 102 as a self-punching inserter, the inserter 102 may be advanced through the anatomy to be positioned proximate a bone. A mallet or other tool may be used to apply a force to the proximal handle end 138 to drive the inserter 102 into the bone 200 until the second laser mark 142 (which defines the insertion zone) may be proximate a surface of the bone. With reference to FIG. 8A, the inserter 102 may be shown driven into the bone 200 to define a bone hole 202. With the inserter 102 in position, the collar 106 may be slid from the first collar position (FIG. 1) to the second collar position (FIG. 7) to enable the surgeon to manipulate the handle 126. The suture retainer 128 may be removed from the handle 126 to uncouple the anchor 108 from the inserter 102. The collar 106 may be slid from the first collar position (FIG. 1) to the second collar position (FIG. 7) to couple the guidewire 104 to the inserter 102. With reference to FIG. 8B, the inserter 102 and the guidewire 104 may be removed from the anatomy. With the inserter 102 removed, the surgeon may pull on the flexible strand 182 to move the sheath 180 into the bunched together configuration to deploy the anchor 108 within the anatomy. With the anchor 108 deployed within the anatomy, the surgeon may use the flexible strand 182 to secure or fix the soft tissue to the bone to promote healing.

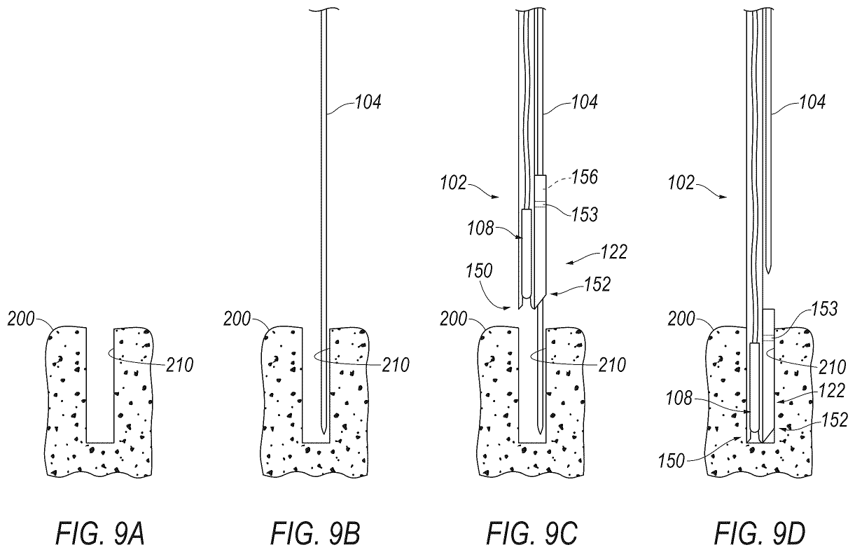

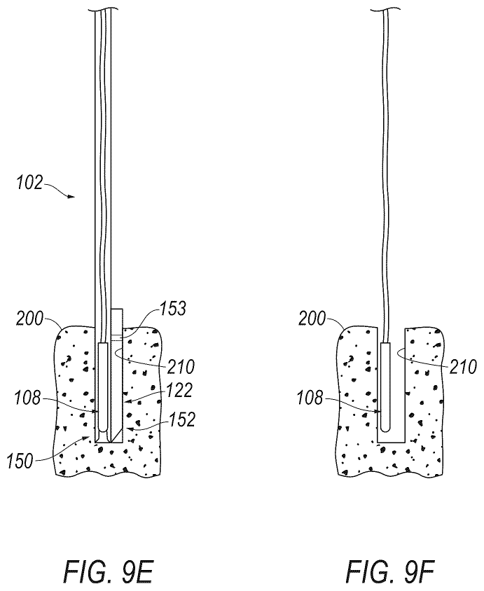

In another example, such as in the example of hard bone, with reference to FIG. 9A, a drill or punch may be used to define a pilot bone hole 210. In this example, with reference to FIG. 9B, the guidewire 104 may be positioned in the pilot bone hole 210 formed by the drill or punch prior to the percutaneous introduction of the inserter 102. With the guidewire 104 coupled to the pilot bone hole 210, with reference to FIG. 9C, the second tine 152 may be aligned with the guidewire 104, and the inserter 102 may be percutaneously introduced into the anatomy over the guidewire 104. The inserter 102 may be advanced over the guidewire 104 until the second laser mark 142 may be aligned with the surface of the bone 200. With the inserter 102 in position, the collar 106 may be slid from the first collar position (FIG. 1) to the second collar position (FIG. 7) to enable the surgeon to manipulate the handle 126. It should be noted that due to the second length L2 of the second tine 152, the guidewire 104 may remain flexible within the pilot bone hole 210, and may be able to move to the side of the pilot bone hole 210 if needed to enable the inserter 102 to deliver the anchor 108 to the center of the pilot bone hole 210.

With reference to FIG. 9D, with the distal end 122 positioned within the pilot bone hole 210, the guidewire 104 may be removed from the anatomy leaving the distal end 122 in position to deploy the anchor 108 as shown in FIG. 9E. The suture retainer 128 may be removed from the handle 126 to uncouple the anchor 108 from the inserter 102. The inserter 102 may be removed from the anatomy, leaving the anchor 108 within the pilot bone hole 210 as shown in FIG. 9F. With the inserter 102 removed, the surgeon may pull on the flexible strand 182 to move the sheath 180 into the bunched together configuration to deploy the anchor 108 within the anatomy. With the anchor 108 deployed within the anatomy, the surgeon may use the flexible strand 182 to secure or fix the soft tissue to the bone to promote healing. In addition, it should be noted that in either of these examples, the inserter 102 may be introduced through a sheath, if desired.

Thus, the anchor inserter system 100 may enable the surgeon to guide the anchor 108 into a predetermined location within the anatomy over the guidewire 104, which may provide for additional accuracy in the placement of the anchor 108 within the anatomy. Further, the use of the guidewire 104 with the inserter 102 may enable the surgeon to insert the anchor 108 without a clear line of sight through tissue, as the guidewire may lead the way through the anatomy.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice, material, manufacturing, and assembly tolerances, and testing capability.

As used herein, the phrase at least one of A, B, and C should be construed to mean a logical (A OR B OR C), using a non-exclusive logical OR, and should not be construed to mean “at least one of A, at least one of B, and at least one of C.”

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. An anchor inserter system comprising:
an inserter having a proximal end portion and a distal end portion, the distal end portion including:
a first tine; and
a second tine opposite the first tine and the second tine is cannulated,
wherein a slot is defined between the first tine and the second tine.

2. The anchor inserter system of claim 1, further comprising an anchor, and a portion of the anchor is coupled within the slot.

3. The anchor inserter system of claim 2, wherein the anchor is a soft anchor.

4. The anchor inserter system of claim 1, wherein the slot is U-shaped.

5. The anchor inserter system of claim 1, wherein the first tine is solid.

6. The anchor inserter system of claim 1, wherein the second tine defines a tine bore extending along an axis parallel to and offset from a longitudinal axis of the inserter.

7. The anchor inserter system of claim 1, further comprising a guidewire, the guidewire being slidably received within the second tine.

8. The anchor inserter system of claim 7, further comprising a collar coupled about a portion of the guidewire.

9. The anchor inserter system of claim 8, wherein the inserter further comprises a graspable portion coupled to the proximal end portion and a shaft interconnecting the proximal end portion and the distal end portion, and the collar is movable between a first position in which the collar is proximate the graspable portion to couple the guidewire to the shaft and a second position in which the collar is spaced apart from the graspable portion.

10. The anchor inserter system of claim 1, wherein the first tine extends for a first length axially along a longitudinal axis of the inserter, the second tine extends for a second length axially along the longitudinal axis of the inserter, and the second length is greater than the first length.

11. An anchor inserter system comprising:
a soft anchor; and
an inserter having a proximal end portion and a distal end portion, the distal end portion including:
a first tine; and
a second tine opposite the first tine, and the second tine is cannulated,
wherein a slot is defined between the first tine and the second tine, and a portion of the soft anchor is received within the slot.

12. The anchor inserter system of claim 11, wherein the slot is U-shaped.

13. The anchor inserter system of claim 11, wherein the first tine is solid.

14. The anchor inserter system of claim 11, wherein the second tine defines a tine bore extending along an axis parallel to and offset from a longitudinal axis of the inserter.

15. The anchor inserter system of claim 11, further comprising a guidewire, the guidewire being slidably received within the second tine.

16. The anchor inserter system of claim 15, further comprising a collar having an interior surface opposite an exterior surface, and the interior surface is coupled about a portion of the guidewire.

17. The anchor inserter system of claim 16, wherein a second portion of the soft anchor extends from the portion along the exterior surface of the collar to the proximal end portion.

18. The anchor inserter system of claim 17, wherein the inserter further comprises a graspable portion coupled to the proximal end portion and a shaft interconnecting the proximal end portion and the distal end portion, and the collar is movable between a first position in which the collar is proximate the graspable portion to couple the guidewire to the shaft and a second position in which the collar is spaced apart from the graspable portion.

19. An anchor inserter system comprising:
a soft anchor having a sheath and a flexible strand;
an inserter having a proximal end portion and a distal end portion interconnected by a shaft, the proximal end portion including a graspable portion, the distal end portion including a first tine and a second tine opposite the first tine, the second tine is cannulated, and a slot is defined between the first tine and the second tine, the sheath being coupled within the slot; and
a collar having an interior surface coupled about a portion of the shaft and an exterior surface, and a portion of the flexible strand extends along the exterior surface of the collar such that the collar is movable relative to the soft anchor.

20. The anchor inserter system of claim 19, further comprising a guidewire, the collar is coupled about a portion of the guidewire and the guidewire is slidably received within the second tine.
